(19) United States
(12) Patent Application Publication
(10) Pub. No.: US 2023/0162617 A1
(43) Pub. Date: May 25, 2023

(54) INDICATION-DEPENDENT NUTRIENT CALCULATION AND PRESERVATION PLATFORM

(71) Applicant: SOCIETE DES PRODUITS NESTLE S.A., Vevey (CH)

(72) Inventor: KURT SEIDENSTICKER, Chicago, IL (US)

(21) Appl. No.: 17/995,127

(22) PCT Filed: Apr. 1, 2021
(86) PCT No.: PCT/EP2021/058559

§ 371 Date: Sep. 30, 2022

Related U.S. Application Data
(60) Provisional application No. 63/005,823, filed on Apr. 6, 2020.

(51) Int. Cl. G09B 19/00; G16H 20/60; G16H 10/65

(52) U.S. Cl. CPC G09B 19/0092; G16H 20/60; G16H 10/65; G16H 10/20

(57) ABSTRACT

Methods and systems for monitoring nutrient levels and recommending dietary intake via a customizable indication-dependent platform are presented. In one embodiment, a method is provided that includes generating, by a computing device having one or more processors and via an application programming interface, an application for assessing a nutritional need and/or nutrient level (e.g., collagen level). The application may prompt the entry of user attributes to assess the nutritional need or nutrient level. The computing device may receive, from a user device associated with a user, the user attributes. Based on the user device and the user attributes, the computing device may store a user profile associated with the user. An assessment of the nutritional need and/or nutrient level may be generated for the user. A recommendation for a dietary intake may be provided to the user via the application.

### TECHNICAL FIELD

Certain aspects of the present disclosure generally relate to a nutrient calculator platform that provides estimated serving sizes and customized fact information about nutritional needs, and further provides a method for preserving the results for future use. Inputs for the nutrient calculator platform include, but are not limited to, information gathered on the indication for which the collagen is being ingest as well as the gender, weight, and age of the proposed user.

### BACKGROUND OF THE INVENTION

A significant portion of public are in need of specific types of nutrients and dietary supplements in order to lead healthy lives, achieve desired cosmetic improvements, or complement food and beverage options in a diet friendly way. While consumers may desire specific physiological, medical, and/or cosmetic improvements to themselves, or may generally seek to be healthy, consumers often lack information about any deficiencies in their nutritional intake. Furthermore, consumers may be unaware of the types of nutritional and dietary supplements available and how to complement them into their daily lives. For example, consumers may lack of knowledge about the possible uses and applications (e.g., recipes) for the dietary supplements.

Various embodiments of the present disclosure address one or more of the shortcomings presented above.

### SUMMARY OF THE INVENTION

The present disclosure presents new and innovative methods and systems for monitoring nutrient levels and recommending dietary intake via a customizable indication-dependent platform. In one embodiment, a method is provided that includes a computing device (e.g., an application server) generating (e.g., via an application programming interface), an application for assessing a nutrient level (e.g., collagen level) and/or nutritional need (e.g., recommended intake of collagen). The nutrient level and/or nutritional need may be user-specific (e.g., wherein a severity may depend on the physiology of the user). Furthermore, the nutritional need (e.g., a severity of a deficiency or overconsumption) may be determined based on the nutrient level. The application, which may be accessible to a user on their user device, may prompt entry of user attributes to assess the nutrient level. The computing device may receive, from the user device associated with the user, one or more user attributes of the user to assess the nutrient level for the user. Based on the user device and the one or more user attributes, the computing device may store a user profile associated with the user. Furthermore, the computing device may generate an assessment of the nutrient level for the user; and may generate (e.g., based on the user profile) a recommendation fora dietary intake for the user. For example, the recommendation may comprise, a product whose contents contribute towards the recommended dietary intake, or a recipe for concocting a preparation includes the recommended dietary intake. In some aspects, the computing device may also display, via the application, a predicted nutrient level based on the recommended dietary intake.

In some aspects, the computing device may send the user device a message, via an application, requesting that the user input physiological data (e.g., heart rate, blood pressure, etc.). The user device may provide the physiological data (e.g., via a wearable and/or accessory device that is communicatively associated with the user device), and this physiological data may be received by the computing device (e.g., as the one or more user attributes). Also or alternatively, the computing device may send to the user device, via an application, a message requesting the user to input a health goal (e.g., desired weight, desired, body mass index, desired body fat percentage, etc.). The user may enter in their health goal, via the application, and the health goal of the user may be received by the computing device. The computing device may determine, based on a comparison of the physiological data and the health goal, the recommendation for the dietary intake for the user.

The computing device may determine a set of user-specific products for the user (e.g., based on the one or more user attributes of the user). The generated recommendation for the dietary intake may include one or more user specific products (e.g., a subset) from the set of user-specific products. In some aspects, the user may be prompted (e.g., via a message received via the application on the user device), to input one or more filters for user-specific products, including but not limited to, an activity level, a food sensitivity, a preferred diet, or a comorbidity. The computing device may filter, from the set of user-specific products, a user-specific product based on the one or more filters inputted by the user.

In some embodiments, the computing device may receive, from the user device, an indication of a user intent. As used herein, a user intent may refer to an intent of the user to use the systems and methods discussed herein to either treat a health condition (e.g., manage obesity, attain weight from an underweight condition, lower blood glucose or cholesterol, overcome a nutrient deficiency, etc.) or to maintain a healthy condition. The application, also referred to as a customizable indication-dependent platform may be customized based on the indicated user intent.

In some embodiments, the customizable indication-dependent platform may be able to remember and/or recognize a user, e.g., based on their user profile and/or stored attributes, and may restore certain configurations of the application accordingly. For example, the computing device may receive, from a second user device associated with the user, one or more user attributes of the user to assess the nutrient level for the user. The computing device may recognize (e.g., by having received the one or more user attributes) the user profile of the user. Thus, the computing device may configure the application accordingly based on the user (e.g., by customizing the application based on any indicated user intent).

In another embodiment, a system is disclosed for monitoring nutrient levels and/or nutritional needs and recommending dietary intake via a customizable indication-dependent platform. The system may comprise one or more processors a memory. The memory stores instructions that, when executed by the one or more processors, cause the system to perform one or more methods described herein. In another embodiment, a non-transitory computer readable medium is disclosed for use on a computer system containing computer-executable programming instructions for monitoring nutrient levels and/or nutritional needs and recommending dietary intake via a customizable indication-dependent platform. The instructions may comprise one or more steps, methods, or processes described herein.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 illustrates a system for monitoring nutrient levels and recommending dietary intake via a customizable indication-dependent platform, according to an embodiment of the present disclosure.

FIG. 2 illustrates an example user profile database according to exemplary embodiments of the present disclosure

FIG. 3 illustrates an example dietary supplement product recommendation engine according to an exemplary embodiment of the present disclosure.

FIG. 4 illustrates a flow diagram of an example method of monitoring nutrient levels and recommending dietary intake via a customizable indication-dependent platform, according to an exemplary embodiment of the present disclosure.

FIGS. 5 and 6 illustrate screenshots of an example user interface of a customizable indication-dependent platform for monitoring nutrient levels and recommending dietary intake, according to an embodiment of the present disclosure.

### DETAILED DESCRIPTION OF THE INVENTION

A significant portion of public are in need of specific types of nutrients and dietary supplements in order to lead healthy lives, achieve desired cosmetic improvements, or complement food and beverage options in a diet friendly way. However, individuals may not necessarily know nutrient deficiencies that they may have. For example, collagen is a ubiquitous naturally occurring protein in the human body, and is responsible for supporting healthy hair, skin, nails, bones, and joints. However, around the age of 25, the human body's production of natural collagen may typically begin to decline. Realizing that nutrients like collagen may be needed, individuals may still be unaware of the types of nutritional and dietary supplements available and how to complement them into their daily lives (e.g., via recipes and other applications).

The present disclosure relates generally to a customizable indication-dependent platform for monitoring nutrient levels and recommending dietary intake. Additionally, the disclosure provides for a method for monitoring and determining various nutritional needs (e.g., recommended individual-specific collagen needs per day). The nutritional needs may depend on, inter alia, the user-specific nutrient levels, a proposed user indication to be treated or prevented, the proposed user's gender, weight, and age, and the proposed user's physical and/or behavioral information. In addition to producing recommendations on dietary intake (e.g., collagen dosages) based upon such inputs, the platform also may present fact information about the proposed user's nutritional needs given their gender, weight and/or age, physical, and/or behavioral data. Furthermore, the platform may recommend various uses (e.g., recipes, products, etc.) for the nutritional and/or dietary supplements to meet the nutritional needs. In some aspects, the platform may leverage wearable biosensors to accurately and precisely monitor a user's health condition to adjust nutritional and/or dietary recommendations.

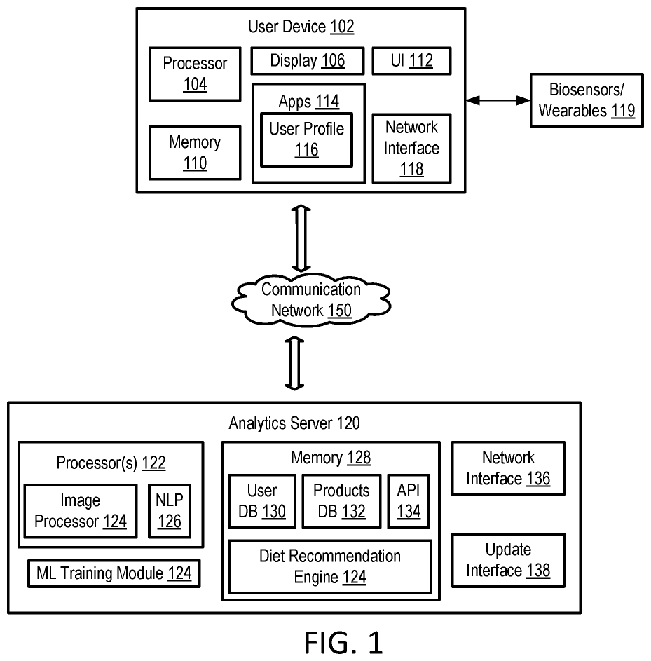

FIG. 1 illustrates a system 100 according to an embodiment of the present disclosure. The system 100 includes a user device 102 associated with a user and an analytics server 120 for performing one or more steps or methods described herein. The user device 102 may be communicatively coupled to one or more biosensors and/or wearable devices 119. Also or alternatively, the biosensors and/or wearables 199 may be a part of a standalone computing device (e.g., at a hospital and/or medical facility). Each of these devices and other external devices (not shown) may be able to communicate with one another over a communication network 150, which may be any wired or wireless network for disseminating information. Examples of the wireless networks may comprise Wi-Fi, a global system for mobile communications (GSM) network, and a general packet radio service (GPRS) network, an enhanced data GSM environment (EDGE) network, 802.5 communication networks, code division multiple access (CDMA) networks, Bluetooth networks or long term evolution (LTE) network, LTE-advanced (LTE-A) network or 5th generation (5G) network. Moreover, each device may include a respective network interface (e.g., network interface 118, 136, 118A, and 146) to facilitate communication through the communication network 150. For example, the respective network interface may comprise a wired interface (e.g., electrical, RF (via coax), optical (via fiber)), a wireless interface, a, modem, etc. Furthermore, each of these devices may include one or more respective processor(s) (e.g., processors 104 and 122) and memory (e.g., memory 110 and 128) The processor may comprise any one or more types of digital circuit configured to perform operations on a data stream, including functions described in the present disclosure. The memory may comprise any type of long term, short term, volatile, nonvolatile, or other memory and is not to be limited to any particular type of memory or number of memories, or type of media upon which memory is stored. The memory may store instructions that, when executed by the processor, can cause the respective device to perform one or more methods discussed herein.

The user device 102 may be implemented as a computing device, such as a computer, smartphone, tablet, smartwatch, or other wearable through which an associated user can communicate with the analytics server 120, e.g., through use of an application 114. The user device 102 may further include a user interface (UI) 112, which may comprise a touch-sensitive display, a touchscreen, a keypad with a display device, or a combination thereof, and may work in relation to a display 106. The UI 112 may allow the first user to view audio, visual, and/or textual information presented by the analytics server 120 via application 114, access and use one or more applications 114, and enter input signals, e.g., by touching and moving icons on the display 106. The display 106 may comprise any medium of outputting visual information (e.g., image, video, etc.). The applications 114 may comprise any program or software to perform the methods described herein. For example, the applications 16 may include an application hosted by the analytics server 120 for monitoring nutrient levels and/or nutritional needs and recommending dietary intake. The user may have a user profile 116 associated with the application, and the user profile 116 may comprise a plurality of user attributes that may be utilized by the analytics server 120. The user attributes may include biographical details about the user (e.g., a user identification, gender, weight, height, etc.), information about the health of the first user (e.g., activity level, comorbidities, health goals, etc.), and dietary needs and preferences. In some embodiments, the user attributes may further include physiological data of the first user. The physiological data may be obtained via one or more biosensors and/or a wearable device that includes the one or more biosensors (e.g., “biosensors/wearables” 119). In some aspects, the biosensors/wearables 119 may be a part of, or may be communicatively linked to, the user device 102. The biosensors/wearables may include, but are not limited to, a glucose monitor, a sphygmomanometer, a heart rate measurement device, and/or devices that measure activity levels (e.g., FITBIT®). The applications 14 may further include one or more social media applications hosted by the social media server 140, and the one or more social media applications may allow the first user to network with the one or more followers.

The analytics server 120 may comprise a local or a remote computing system for requesting and receiving information received from the user device 102; processing information associated with a user or a follower; learning from various user-specific data concerning nutrient levels and user attributes to train machine learning models for predicting nutrient levels, nutritional needs, and dietary intake recommendations; generating a list of recommended products and uses of the products; and sending recommendations.

The one or more processors 122 of the analytics server 120 may include an image processor 124 and a natural language processor 126. The image processor 124 may digitally process image data produced by the user device 102 to avoid noise and other artifacts, and prepare such image data for the recognition of texts and physical objects. The natural language processor 126 may be used to recognize texts, and determine meaning from texts captured from the image data. The texts may include the identifiers of user attributes and recognizable products (e.g., product name, company name, etc.). The image processor and/or natural language processor may rely on stored machine learning models from the machine learning module 124 to recognize, from image and text data, information for various user attributes (e.g., age and weight information from text in natural language, calories consumed per day from images of food, health conditions from text describing a health condition in natural language, etc.). In one aspect, the machine learning module 124 may utilize reference image data to perform supervised learning to be able to accurately identify relevant user attributes. Furthermore, the machine learning training module 124 may also be used to train models to calculate and/or predict a nutrient level based on reference training data. In this example, the reference training data may comprise feature vectors that each comprises values based on and corresponding to a set of user attributes for a plurality of individual users. The feature vectors may be associated with known nutrient levels for those plurality of individual users, and may be trained as a supervised machine learning model.

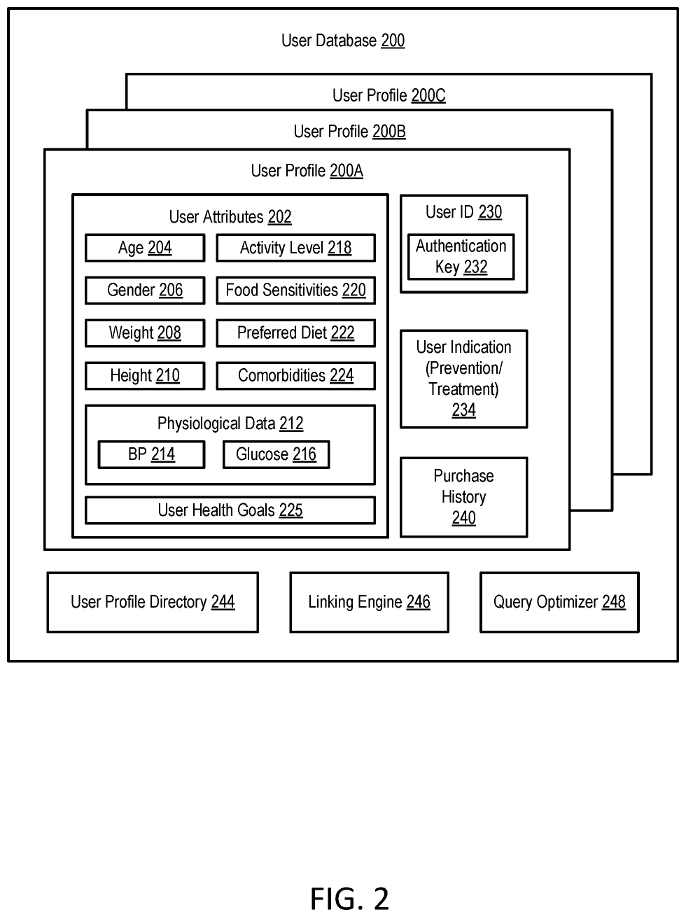

The memory 128 of the analytics server 120 may further include a user database 130, a products database 132, and an application program interface 134. The user database 130 may store the respective user profiles associated with the users of the systems and methods presented herein (e.g., including the user associated with user device 102). FIG. 2 shows an example embodiment of the user database 130 in further detail, as will be described further below. The products database 132 may store information about a plurality of recognizable products. The recognizable products can provide nutritional or dietary supplements, and can be the subject of various multimedia content. The products database 132 may list nutritional information for each product, uses of the product within recipes, and special instructions and warnings considering their usage. The products may include food and beverage products associated with nutritional and dietary supplements. An example product may include collaged infused edible products, such as collaged peptide powders, collaged-base additive for beverages, and collagen infused water. The memory may also include an application program interface (API) 134 that host, manage, or otherwise facilitate one or more applications in the user device 102 (e.g., application 114). For example, the API 134 may manage an application that enables the monitoring nutrient levels and recommending dietary intake. The diet recommendation engine 124 may comprise one or more programs, applications, or implementations that utilize the user database 130 and products database 132 to generate appropriate recommendations and discounts associated with a set of one or more products for a given user (e.g., user).

The analytics server 120 may further include an update interface 138. The update interface 138 may comprise a database management program or application for managing one or more databases (e.g., user database 130, products database 132, etc.), such as via create, read, update, or delete (CRUD) functions. In some aspects, the update interface 138 may allow external devices (e.g., user device 102) to updated one or more databases, e.g., such as when a new user would like to sign up to an application for monitoring nutrient levels and recommending dietary intake.

FIG. 2 illustrates an example user profile database 200 according to exemplary embodiments of the present disclosure. As discussed previously, the user profile database 200 may be a component of the analytics server 120. The user profile database 200 may allow the analytics server 120 to personalize diet and nutritional intake recommendations for the user with respect to various products and help the user to achieve health goals, among other functions. The user database 200 may comprise a plurality of user profiles 200A-200C corresponding to a plurality of users of the application.

For example, user profile 200A, which may be representative of the plurality of user profiles, may include a plurality of user attributes 122. For example, the user attributes 122 may be populated by information regarding one or more of age 204, gender 206, weight 208, height 210, activity level 212, food sensitivities 220, preferred diet 222, co-morbidities 220, physiological data 212 (e.g., blood pressure 214, glucose levels 216, etc.) and user health goals 225. Some examples of food sensitivities 212 include lactose, eggs, nuts, shellfish, soy, fish, and gluten sensitivities. Some non-limiting examples of a preferred diet 214 includes vegetarian, vegan, Mediterranean, kosher, halal, paleo, low carb, and low fat diets. Other non-limiting examples of physiological data 212 may include oxygen levels, heart rate, body temperature, body fat percentage, etc. Some non-limiting examples of co-morbidities 220 include diabetes, obesity, high blood pressure, high cholesterol, celiac, and heartburn. In some aspects, the user may be prompted, via application 114, to provide physiological data, e.g., through a wearable biosensor device 119. In some aspects the user may be prompted, e.g., via a message on application 114, to enter their health goals, e.g., via user interface 112. Some non-limiting examples of user health goals may include a target weight, a target physiological data (e.g., a target blood pressure, a target glucose level, a target body fat percentage, etc.).

The user profile 200A may further include a stored user ID 230 associated with the user and an authentication key 232. The user ID 230 may be used to identify the user, the user device 102 (or follower device 102A-102C) of the user, or a social media profile associated with the user. The authentication key may include a public and/or a private key generated by the authentication module 139 for verifying a user, e.g., to enlist a new user into the application for monitoring nutrient levels and recommending dietary intake.

Furthermore, the user profile 200A may track and record the purchase history 240 of the user, e.g., as it relates to products from product database 132. In some aspects, the purchase history may be able to determine various user attributes for the user, determine nutrient levels based on consumption of a purchased product, or generate a list of user-specific products based on purchasing behavior.

The user database 200 may also include a user profile directory 244 that maps the various user profiles 200A-200C in the user database 200, and a linking engine 246 that links various data structures in the user database 200 (e.g., user profiles 200A-200C) to data structures in other databases (e.g., products database 132). Furthermore a query optimizer 248 may allow external components and devices to more efficiently and accurately retrieve information from the user database 200.

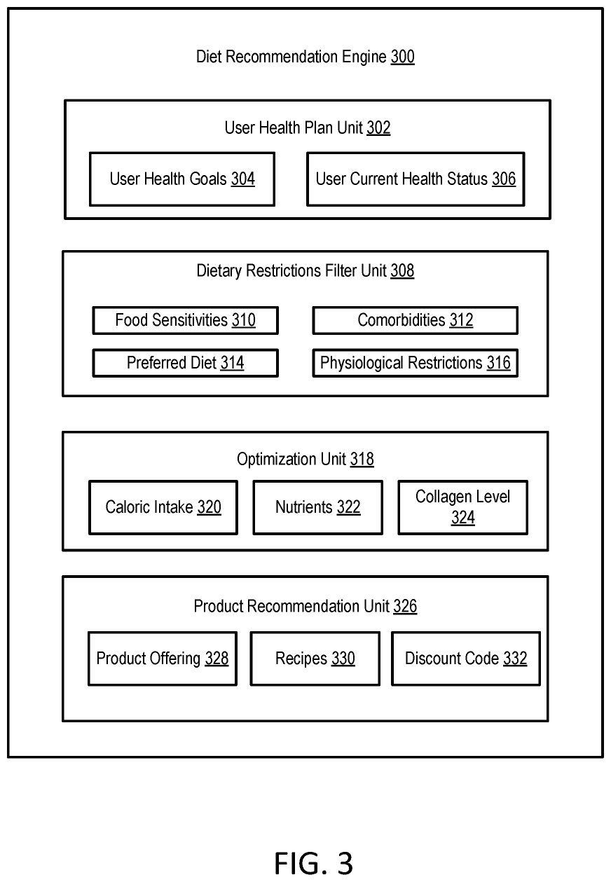

FIG. 3 illustrates an example dietary supplement product recommendation engine (“diet recommendation engine”) 300 according to exemplary embodiments of the present disclosure. As previously discussed, the diet recommendation engine 300 may comprise one or more programs, applications, or implementations in the analytics server 120 that utilize user attributes from the user database 130 and product information from the products database 132 to generate appropriate recommendations and discounts associated with a set of one or more products for a given user.

In an example embodiment, the diet recommendation engine 300 may comprise a user health plan unit 302, a dietary restrictions filter unit 308, an optimization unit 318, and a product recommendation unit 326. The user health plan module 302 may comprise instructions for computing a health plan for the user based on the user health goals 304 retrieved from a user profile of the user (e.g., from user profile database 200)) and the user current health status 306. The user current health status 306 may comprise a data structure storing various user attributes that indicate the current health of a user. For example, the user current health status 306 may comprise a calculated body mass index (BMI) for a given user based on the height 210, weight 208, gender 206, and age 204 of the given user. The user health goal may be a desired BMI that the user would like to achieve. The user health plan module 302 calculates the difference in weight that may be necessary to achieve that. Furthermore, based on an indicated activity level 218, the use health plan module 302 may calculate, for example, the dietary needs of the given user to achiever the user health goals 304 indicated by the user.

The dietary filter restrictions unit 144, which may restrict the results provided by the product recommendation unit 326, may comprise filters for one or more of food sensitivities 310, preferred diets 312, comorbidities 316, and physiological restrictions 316. The optimization unit 318 may contain optimization rules based on one or more of caloric intake 320, specific nutrients 322, and/or collagen level 324.

The diet recommendation engine 300 may further include a product recommendation unit 326 that may store programs and instructions for generating a list of products (e.g., product offerings 328) that pertain to the user (e.g., “user-specific products”) based on the user attributes provided, assessments rendered by the user health plan module 302, dietary filter restrictions 308, and optimization unit 318. The product offerings 326 may include user-specific products comprising food and beverage products associated with specific nutrients (e.g., proteins (e.g., collagen), vitamins, minerals, fat, water, etc.). The product offerings may be identifiable from the products database 132. The product recommendation unit 326 may further generate a list of recommended recipes 330 based on the product offerings. Furthermore, the product recommendation unit 326 may generate a discount code 332 to allow the user to purchase and/or obtain a product from the product offering 328 at a discounted price, or for free.

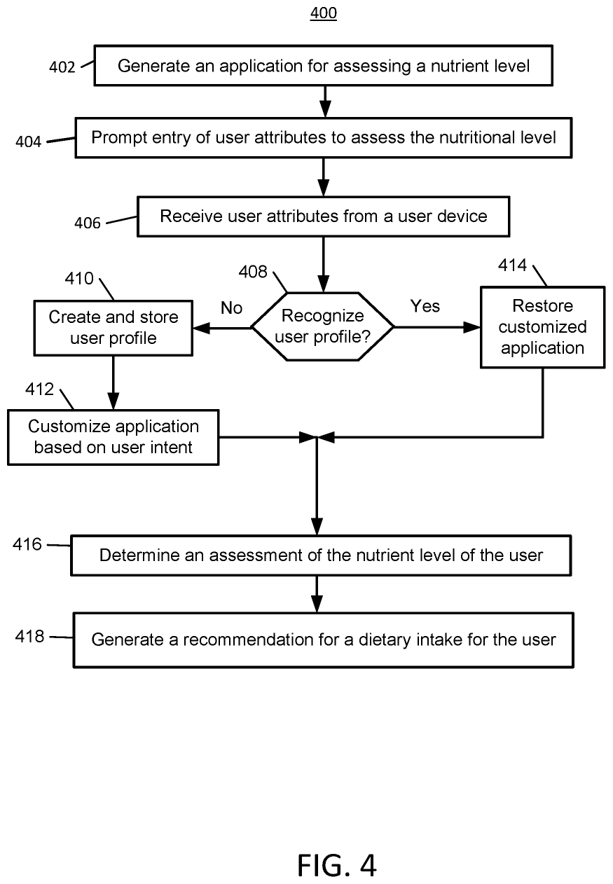

FIG. 4 illustrates a flow diagram of an example method 400 of monitoring nutrient levels and recommending dietary intake, according to an exemplary embodiment of the present disclosure. Method 400 may be performed by one or more processors of the analytics server 120. Furthermore, while method 400 may concern the ability for a user of the application 114 to allow the analytics server 120 monitor the user's nutritional levels, determine nutritional needs, and recommend dietary intake, method 400 may be implemented by other users (e.g., in parallel), via the respective user devices of the other users.

Referring to method 400, step 402 may include the analytics server generating an application for assessing a nutrient level (e.g., as in step 402). For example, analytics server 120 may generate application 114, which may be hosted by API 134, and the application may be accessible to users via their respective user devices. The application may be web browser enabled and/or as a mobile application accessible on mobile platforms.

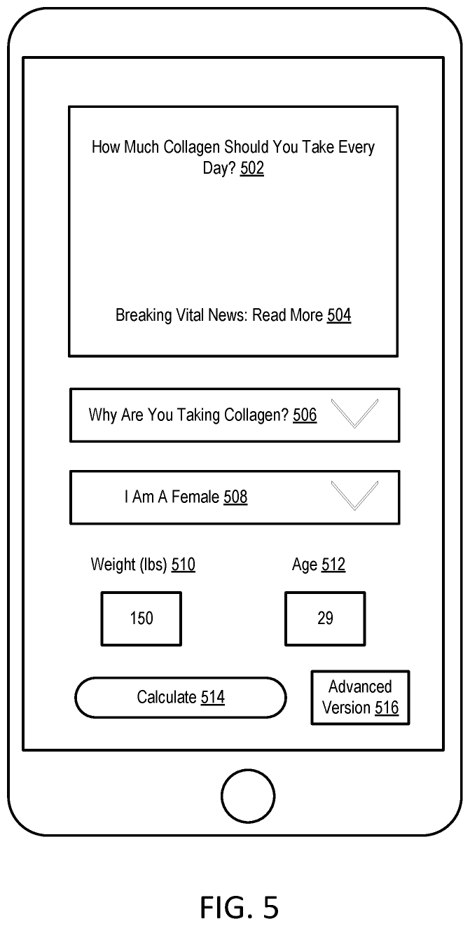

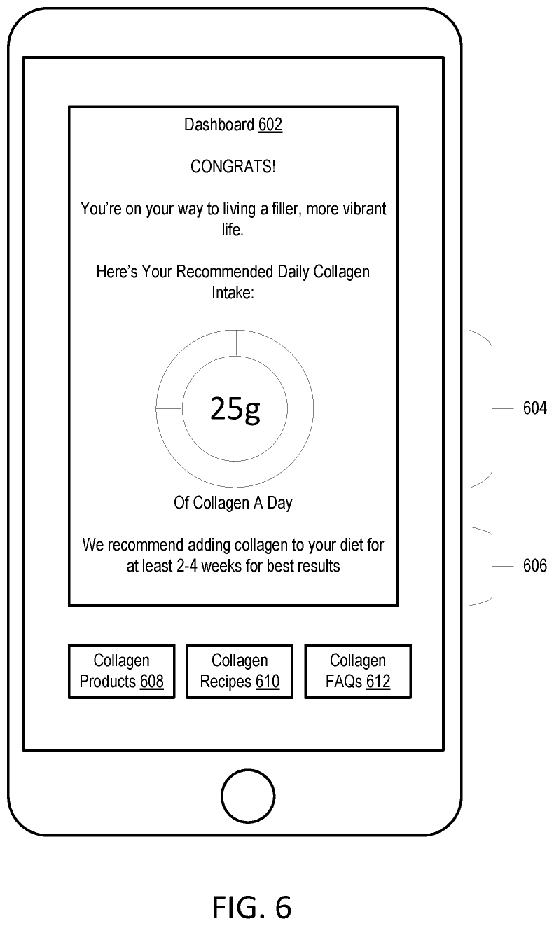

The application may present visual and/or textual information that may invite users to determine their nutrient levels through an interactive tool presented herein, may encourage users to learn more about the nutrient, and may recommend dietary intake solutions. Example screenshots of the user interface of the application is shown in FIGS. 5 and 6.

The application may also, as shown in step 404, prompt users to enter user attributes to assess their nutrient level. For example, a dashboard on the application may ask a set of questions for users to answer to determine a level of a specific nutrient (e.g., in the collagen).

At step 406, the analytics server 120 may receive responses for the user attributes from a user device. For example, a user that is accessing a web enabled version of the application may decide to find out how much their collagen levels are and accordingly enter, into the application, responses to questions. The entered responses may be processed and recognized as user attributes by the analytics server 120.

As discussed previously, the user attributes may include biographical details about the user (e.g., a user identification, gender, weight, height, etc.), information about the health of the first user (e.g., activity level, comorbidities, health goals, etc.), dietary needs and preferences, and physiological data. In some aspects, prompting users to enter a user attribute may involve prompting the user to connect to a biosensor and/or wearable device (e.g., biosensors/wearables 119) to enter physiological data. Also or alternatively, an entry of a user attribute may be via an image, audio, and/or a video upload. For example, a patient data sheet may be scanned and uploaded on to the application 114, and the natural language processor 126 may parse and recognize user attributes pertaining to comorbidities, underlying health conditions, and biographical details.

At step 408, the analytics server 120 may determine whether it recognizes a user profile, e.g., based on the entered user attributes or based on the user device interacting with the application as user attributes are entered. For example, the analytics server may track user devices that interact with the application (e.g., by storing device identifiers), and may recognize when the same devices come back to the application. Also or alternatively, when a user enters responses for user attributes, a threshold number of same responses as another user's responses may cause the analytics server 120 to identify the instant user and the another user as the same user.

If the analytics server 120 cannot recognize the user, the analytics server 120 may create a store a new user profile based on the entered responses for the user attributes. In some aspects, the analytics server 120 may prompt the user, e.g., via a message sent to the user device 102 via the application 114, to enter an intent for using the application (“user intent”). The user may thus enter an indication of the user intent via the application. The user intent may indicate, for example, an intent to treat a health condition. Also or alternatively, the user intent may indicate an intent to prevent the health condition. Also or alternatively, the user intent may indicate an intent to maintain a healthy condition.

At step 412, depending on the expressed intent, the analytics server 120 may customize the application accordingly. For example, as will be discussed further herein, the analytics server 120 may provide adjust dietary intake recommendations, news stories, articles, promotional and/or marketing material based on the user intent.

If, at step 408, the analytics server 120 does recognize a user profile based on entered responses for user attributes, the analytics server 120 may, at step 414, restore a previously customized application based on the user profile. For example, the user associated with the user profile may have previously accessed the application and indicated a certain user intent, which caused the analytics server 120 to have customized the application for the user.

At step 416, the analytics server 120 may determine an assessment of the nutrient level of the user. The analytics server may convert responses of the user for the various user attributes to values. The values for a set of user attributes may be compared to existing models to determine or extrapolate a nutrient level for the user. In some aspects, the values for the set of user attributes may be used to create a feature vector. A machine learning model may be identified that had been trained using the same or similar set of user attributes for training datasets having known nutrient levels. The feature vector may be inputted into the identified machine learning model to determine an assessment of the nutrient level for the user. In some aspects, the analytics server 120 may determine how much nutrient the user may need (e.g., How much collagen the user should take per day) based on the determined nutrient level. In some aspects, the assessment may include estimations of one or more of the approximate amount or percent of the nutrient in the user or the approximate amount or percentage that the nutrient is above or below a recommended amount for the user.

At step 418, the analytics server 120 may generate a recommendation for a dietary intake for the user. The recommendation for the dietary intake may include an amount of nutrient that the user may need on a periodic basis (e.g., daily, weekly, monthly, etc.) to achieve recommended levels of the nutrient, and/or to meet a health goal that the user may have selected and entered via the application. Furthermore, the recommendation for the dietary intake may include a recommendation of one or more user-specific products (e.g., food and/or beverages) known to have nutrients that will help the user reach the recommended nutrient level.

In some embodiments, the analytics server 120 may further assist the first user in achieving their health goals. For example, the user may have input and/or upload, via UI 112, various user attributes into the application 114 (e.g., at step 406). The analytics server 120 may use these user attributes to develop a plan for the user to achieve his or her desired health goal, and offer user-specific products that help the user meet the desired health goal. For example, the analytics server 120 may determine, based on the user attributes, a set of user-specific products. As previously discussed in relation to FIG. 3, the user-specific products may be based on an assessment of products that can help the user meet their desired health goals, and may be filtered (e.g., based on dietary restrictions), and optimized accordingly.

In some aspects, user attributes comprising real-time physiological data (e.g., heart rate, blood pressure, glucose level, etc.) may enable a more accurate monitoring of nutritional needs and a more effective product offering for the user. For example, the analytics server may prompt the user to input physiological data by sending, via application 114 on the mobile device 102, a message requesting the user to input physiological data. For example, a user desiring to improve one's athletic performance over time may be prompted to provide a heart rate. A biosensor or wearable associated with or communicatively linked to the user device 102 may be used by the user to provide such physiological input. The analytics server 120 may then receive, from the mobile device, the physiological data.

The user's health goal is yet another user attribute that can be used to determine a set of user-specific products for the first user. In one aspect the analytics server 120 may send, via the application 114 on the user device 102, a message requesting that the user to input their health goal. Thereafter, the analytics server 120 may receive the health goal indicated by the user. Based on the health goal, the analytics server may determine a set of user-specific products. In further aspects, the user's stated health goal may compared with the user's current physiological data to determine the set of first user-specific products.

FIGS. 5 and 6 illustrate screenshots of an example user interface of a customizable indication-dependent platform for monitoring nutrient levels and recommending dietary intake, according to an embodiment of the present disclosure. The example screenshots may be based on a graphical user interface provided by application 114, and may be viewable by the user on user device 102. The customizable indication-dependent platform may be application 114 that may be accessible to various users (e.g., via their respective user devices) and may be managed by analytics server 120 via API 134.

Referring now to FIG. 5, the example user interface may invite the user to determine whether there is a specific nutrient need (e.g., “How Much Collagen Should You Take Every Day” 502). To entice or encourage the user to participate in having their nutrient level assessed, the example user interface may also provide news alerts, stories, articles, and/or other multimedia content that informs the user of the benefit of the specific nutrient (e.g., “Breaking Vital News: Read More” 504). The example user interface may also provide functionalities (e.g., data fields, tabbed options) for the user to select responses for various user attributes (e.g., “Why Are You Taking Collagen” 506, “I Am A Female” 508, Weight 508, and age 512. Some user attributes, such as “Why Are You Taking Collagen” may be used by the analytics server 120 to determine user intent and to customize the user interface of the application accordingly. The user can then select calculate 514 to determine the nutrient level. In some aspects, the user may be able to avail themselves of a more accurate or comprehensive assessment of a nutrient level and/or a dietary intake recommendation through an advanced version 516 of the application. The advanced version may prompt entry of response for significantly more user attributes.

FIG. 6 illustrates another screenshot of an example user interface of the customizable indication-dependent platform for monitoring nutrient levels and recommending dietary intake, according to an embodiment of the present disclosure. Specifically, FIG. 6 shows an example user interface after the user has opted to have the application determine their nutrient level (e.g., by clicking calculate 514 in FIG. 5). As shown in FIG. 6, the user may be led to a dashboard 602 that praises the user for willing to devote themselves to a healthier future by way of the application for monitoring nutrient levels and recommending dietary intake. For example, the dashboard 502 indicates the recommended daily collagen intake for the user as “25 g of collagen a day.” Furthermore, the application offers a dietary intake recommendation of adding collagen to the user's diet for at least 2-4 weeks for best results. As options to meet this dietary intake recommendation, the user may be able to access an inventory of, or purchase, collagen products 608; browse collagen-based recipes (e.g., Collagen Recipes 610); and/or learn more about collagen generally (e.g., via Collagen FAQ 612).

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. A method of monitoring collagen levels and recommending dietary intake via a customizable indication-dependent platform, the method comprising:
generating, by a computing device having one or more processors and via an application programming interface, an application for assessing a user-specific collagen level, wherein the application prompts entry of user attributes to assess the user-specific collagen level;
receiving, by the computing device and from a user device associated with a user, one or more user attributes of the user to assess the user-specific collagen level for the user;
storing, based on the user device and the one or more user attributes, a user profile associated with the user;
generating, by the computing device, an assessment of the user-specific collagen level for the user; and
generating, based on the user profile, a recommendation for a dietary intake for the user.

2. The method of claim 1, wherein the receiving the one or more user attributes comprises:
sending, to the user device via the application, a message requesting the user to input physiological data; and
receiving, from the user device, the physiological data.

3. The method of claim 2, further comprising:
sending, to the user device via the application, a message requesting the user to input a health goal;
receiving, from the user device, the health goal; and
determining, based on a comparison of the physiological data and the health goal, the recommendation for the dietary intake for the user.

4. The method of claim 1, further comprising:
determining, based on the one or more user attributes, a set of user-specific products, wherein the generating the recommendation for the dietary intake comprises one or more user specific products from the set of user-specific products.

5. The method of claim 4, further comprising:
sending, to the user device via the application, a message requesting the user to input one or more of an activity level, a food sensitivity, a preferred diet, or a comorbidity; and
filtering, from the set of user-specific products, a user-specific product based on the one or more of the activity level, the food sensitivity, the preferred diet, or the comorbidity.

6. The method of claim 1, further comprising:
receiving, from the user device via the application, an indication of a user intent, the user intent comprising one of a first intent to treat a health condition, or a second intent to prevent the health condition;
customizing, by the computing device, the application based on the indication of the user intent.

7. The method of claim 1, further comprising:
receiving, by the computing device and from a second user device associated with the user, the one or more user attributes of the user to assess the user-specific collagen level; and
recognizing, by the computing device and based on the one or more user attributes, the user profile of the user.

8. A system for monitoring nutritional needs and recommending dietary intake, the system comprising:
one or more processors; and
memory storing instructions that, when executed by the processors, cause the system to:
generate, via an application programming interface, an application for assessing a nutritional need, wherein the application prompts entry of user attributes to assess the nutritional need;
receive, from a user device associated with a user, one or more user attributes of the user to assess the nutritional need for the user;
store, based on the user device and the one or more user attributes, a user profile associated with the user;
generate an assessment of the nutritional need for the user; and
generate, based on the user profile, a recommendation for a dietary intake for the user.

9. The system of claim 8, wherein the instructions, when executed, cause the system to receiving the one or more user attributes by:
sending, to the user device via the application, a message requesting the user to input physiological data; and
receiving, from the user device, the physiological data.

10. The system of claim 9, wherein the instructions, when executed, further cause the system to:
send, to the user device via the application, a message requesting the user to input a health goal;
receive, from the user device, the health goal; and
determine, based on a comparison of the physiological data and the health goal, the recommendation for the dietary intake for the user.

11. The system of claim 8, wherein the instructions, when executed, cause the system to:
determine, based on the one or more user attributes, a set of user-specific products, wherein the generating the recommendation for the dietary intake comprises one or more user specific products from the set of user-specific products.

12. The system of claim 11, wherein the instructions, when executed, further cause the system to:
send, to the user device via the application, a message requesting the user to input one or more of an activity level, a food sensitivity, a preferred diet, or a comorbidity; and
filter, from the set of user-specific products, a user-specific product based on the one or more of the activity level, the food sensitivity, the preferred diet, or the comorbidity.

13. The system of claim 8, wherein the instructions, when executed, further cause the system to:
receive, from the user device via the application, an indication of a user intent, the user intent comprising one of a first intent to treat a health condition, or a second intent to prevent the health condition;
customize the application based on the indication of the user intent.

14. The system of claim 8, wherein the instructions, when executed, further cause the system to:
receive, by the computing device and from a second user device associated with the user, the one or more user attributes of the user to assess the nutritional need for the user; and
recognize, by the computing device and based on the one or more user attributes, the user profile of the user.

15. A non-transitory computer readable medium for use on a computer system containing computer-executable programming instructions for monitoring nutrient levels and recommending dietary intake via a customizable indication-dependent platform, the instructions comprising:
generating, by a computing device having one or more processors and via an application programming interface, an application for assessing a nutrient level, wherein the application prompts entry of user attributes to assess the nutrient level;
receiving, by the computing device and from a user device associated with a user, one or more user attributes of the user to assess the nutrient level for the user;
storing, based on the user device and the one or more user attributes, a user profile associated with the user;
determining, by the computing device, an assessment of the nutrient level for the user;
generating, based on the user profile, a recommendation for a dietary intake for the user; and
displaying, via the application and based on the recommendation for the dietary intake, a predicted nutrient level for the user.

16. The non-transitory computer readable medium of claim 15, wherein the receiving the one or more first user attributes comprises:
sending, to the user device via the application, a message requesting the first user to input physiological data; and
receiving, from the user device, the physiological data.

17. The non-transitory computer readable medium of claim 16, wherein the instructions further comprises:
sending, to the user device via the application, a message requesting the first user to input a health goal;
receiving, from the user device, the health goal; and
determining, based on a comparison of the physiological data and the health goal, the recommendation for the dietary intake for the user.

18. The non-transitory computer readable medium of claim 15, wherein the instructions further comprises:
determining, based on the one or more user attributes, a set of user-specific products, wherein the generating the recommendation for the dietary intake comprises one or more user specific products from the set of user-specific products.

19. The non-transitory computer readable medium of claim 18, wherein the instructions further comprises:
sending, to the user device via the application, a message requesting the user to input one or more of an activity level, a food sensitivity, a preferred diet, or a comorbidity; and
filtering, from the set of user-specific products, a user-specific product based on the one or more of the activity level, the food sensitivity, the preferred diet, or the comorbidity.

20. The non-transitory computer readable medium of claim 15, wherein the instructions further comprises:
receiving, from the user device via the application, an indication of a user intent, the user intent comprising one of a first intent to treat a health condition, or a second intent to prevent the health condition;
customizing, by the computing device, the application based on the indication of the user intent.
